(19) United States
(12) Patent Application Publication
(10) Pub. No.: US 2006/0149193 A1
(43) Pub. Date: Jul. 6, 2006

(54) High pressure liquid jet ablation of tissue and apparatus

(75) Inventor: Robert C. Hall, Fort Wayne, IN (US)

Correspondence Address: PEARNE & GORDON LLP, 1801 EAST 9TH STREET, SUITE 1200, CLEVELAND, OH, 44114-3108, US

(73) Assignee: Biomec, Inc., Cleveland, OH (US)

(21) Appl. No.: 11/325,991

(22) Filed: Jan. 5, 2006

Related U.S. Application Data
(60) Provisional application No. 60/641,617, filed on Jan. 5, 2005.

(51) Int. Cl. A61M 31/00

(52) U.S. Cl. 604/275

(57) ABSTRACT

Methods and devices of treating an area of tissue of a patient are provided wherein the area of tissue includes a first tissue layer and a second tissue layer. The methods comprise the step of providing a liquid distribution source including a quantity of liquid and a nozzle in liquid communication with the liquid distribution source. The method further comprises the step of positioning the nozzle with respect to the area of tissue and disbursing the quantity of liquid through the nozzle with a velocity sufficient to substantially remove the first tissue layer without significant injury to the second tissue layer.

### CROSS-REFERENCE TO RELATED APPLICATION

The present invention claims the benefit of U.S. Provisional Application No. 60/641,617 filed Jan. 5, 2005, the entire disclosure which is herein incorporated by reference.

### FIELD OF THE INVENTION

The present invention relates to a surgical procedure and apparatus, and more particularly, to high pressure liquid jet ablation of tissue and apparatus.

### BACKGROUND OF THE INVENTION

Numerous therapies are emerging for treatment of abnormal growth in the esophagus to help prevent development of cancer in the esophagus. Abnormal growth in lower portions of the esophagus, also known as Barrett's Esophagus (intestinal columnar epithelium) is often a precursor to adenocarcinoma of the esophagus. Abnormal growth in upper portions of the esophagus can also lead to squamous cell carcinoma.

Known treatment techniques are believed to involve ineffective removal of the effected esophageal mucosa layer. Existing methods involve procedures with unacceptable depth control that results in unintentional injury to otherwise healthy underlying submucosal layers. Accordingly, there is a need for methods and apparatus adapted to provide selective removal of damaged tissue in the gastrointestinal tract while minimizing or preventing injury to the underlying submucosal layers.

### SUMMARY OF THE INVENTION

Accordingly, it is an aspect of the present invention to obviate problems and shortcomings of conventional surgical procedures and apparatus.

In accordance with one aspect, a method of treating an area of tissue of a patient is provided wherein the area of tissue includes a first tissue layer and a second tissue layer. The method comprises the step of providing a liquid distribution source including a quantity of liquid and a nozzle in liquid communication with the liquid distribution source. The method further comprises the step of positioning the nozzle with respect to the area of tissue and disbursing the quantity of liquid through the nozzle with a velocity sufficient to substantially remove the first tissue layer without significant injury to the second tissue layer.

In accordance with another aspect, a method of treating an esophagus of a patient is provided. The method comprises the step of providing a liquid distribution source including a quantity of liquid, providing a high-pressure source configured to apply pressure to the quantity of liquid, and providing a nozzle in liquid communication with the liquid distribution source. The method further comprises the step of positioning the nozzle with respect to the area of the esophagus and disbursing the quantity of liquid through the nozzle with a velocity sufficient to substantially remove a mucosa layer of the esophagus without significant injury to the submucosal layer of the esophagus.

In accordance with still another aspect, a device for treating an area of tissue of a patient is provided. The device comprises a liquid distribution source including a quantity of liquid, a high-pressure source configured to apply pressure to the quantity of liquid, and a nozzle in liquid communication with the quantity of liquid. The device is configured to disburse the quantity of liquid through the nozzle with a velocity sufficient to substantially remove tissue from a patient.

### BRIEF DESCRIPTION OF THE DRAWINGS

The foregoing and other aspects of the present invention will become apparent to those skilled in the art to which the present invention relates upon reading the following description with reference to the accompanying drawings, in which:

FIG. 1 is a schematic illustration of an apparatus for high pressure liquid jet ablation of tissue;

FIG. 2 is a schematic illustration of the apparatus of FIG. 1 being used for high pressure liquid jet ablation of tissue; and

FIG. 3 is an enlarged schematic illustration of portions of the apparatus taken at view 3 of FIG. 2.

### DETAILED DESCRIPTION OF EXAMPLE EMBODIMENTS

Certain terminology is used herein for convenience only and is not to be taken as a limitation on the present invention. Further, in the drawings, the same reference numerals are employed for designating the same elements.

Devices herein are adapted to provide selective removal of mucosal linings including damaged or otherwise abnormal tissue in the gastrointestinal tract. For example, devices herein may be used to provide selective removal of mucosal linings in the upper or lower portion of the esophagus to prevent development of squamous cell carcinoma or adenocarcinoma of the esophagus.

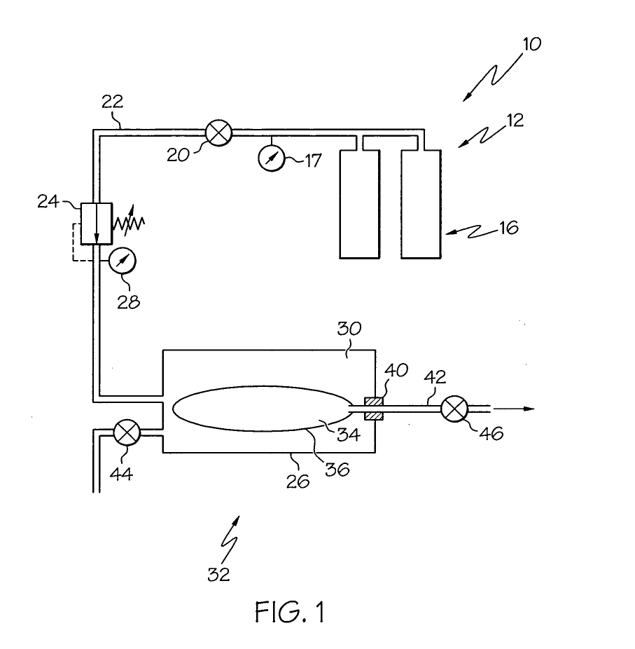

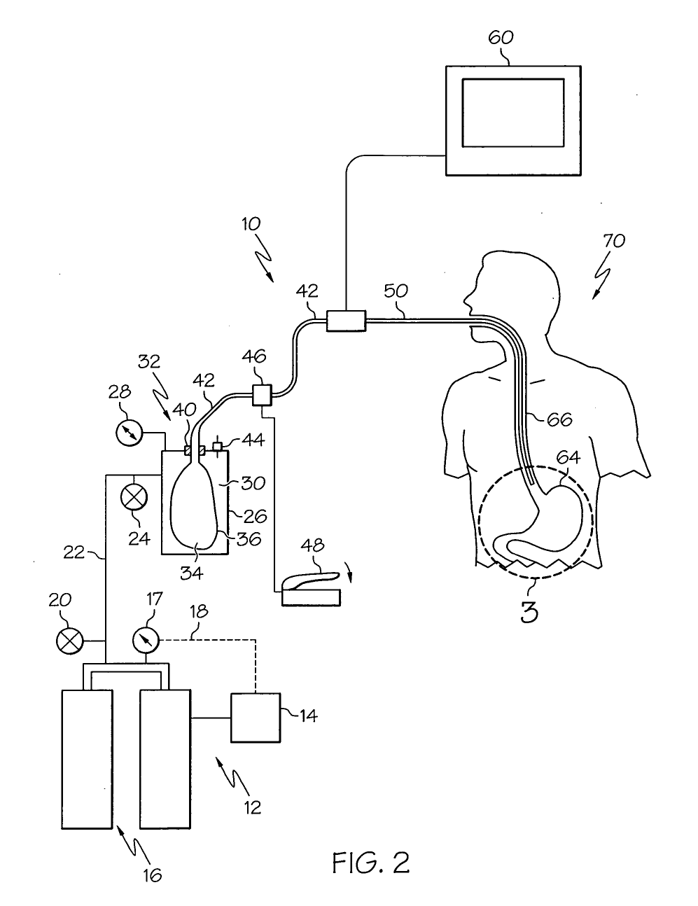

FIGS. 1 and 2 depict schematic illustrations of portions of an exemplary device 10 adapted to be endoscopically deployed into an esophagus 66 of a patient 70 and including a high-pressure liquid jet system that is designed to selectively remove tissue during a surgical procedure. In one particular application, the high-pressure liquid jet system can be designed to facilitate surgical procedures on the esophagus wherein the esophageal mucosa layer 68 is removed while inflicting only minor trauma to the underlying collagen rich submucosal layer 69.

As shown, the device 10 includes a high-pressure source 12. In one example, the high-pressure source 12 includes a pump 14 in communication with a high pressure reservoir 16, such as two high pressure air tanks. A tank pressure gauge 17 may be provided to monitor the pressure within the high pressure reservoir 16. A feedback signal 18 may also be provided to accommodate a feedback control system to automatically maintain the pressure reservoir 16 within a desired pressure range or near a predetermined pressure. In other embodiments the high-pressure source may simply comprise a pump provided in direct communication with a pressure chamber 26 including a quantity of liquid 34 (e.g., saline solution) in a container 36 surrounded by pressurized gas. Therefore, the pump may be provided without necessarily requiring a pressure reservoir.

The high-pressure source 12 is in fluid communication with a high pressure line 22 by way of a control valve 20. The control valve 20 provides an “on-off” function to fully pressurize the pressure line 22 with pressurized fluid from the high pressure reservoir 16 or, alternatively, to substantially or entirely isolate the pressure line 22 from the high pressure reservoir 16. A pressure regulator valve 24 is further provided to control the pressure within a pressure chamber 26. A pressure gauge 28 can be placed in communication with an interior 30 of the pressure chamber 26 or can be placed in communication downstream of the pressure regulator valve 24 to allow observation of the pressure within the interior 30 of the pressure chamber 26. The high-pressure source 12 is adapted to raise the pressure within the interior 30 of the pressure chamber 26 to various levels of pressure. In exemplary embodiments, the interior 30 of the pressure chamber 26 may be pressurized to a selected pressure level, for example, several hundred pounds per square inch.

The device 10 further includes a liquid distribution source 32 that includes the pressure chamber 26. A quantity of liquid 34, such as a saline solution, is contained within a pressure sensitive container 36. In one example, the pressure sensitive container 36 comprises a flexible bag positioned within the interior 30 of the pressure chamber 26. The pressure chamber 26 includes an opening provided with a hermetic seal 40 to permit passage of a tube 42 for transmitting pressurized fluid from the pressure sensitive container 36 to the surgical site. The pressure chamber 26 might also include a pressure release valve 44 to limit the maximum pressure within the pressure chamber. Thus, in an overpressure condition, the pressure release valve 44 might release pressurized fluid from the interior 30 until the pressure within the interior 30 drops to an acceptable level of pressure. The pressure release valve 44 can also be designed to reduce pressure in the interior 30 when the device 10 is not in use. For example, when the device 10 is powered down, the pressure release valve 44 can release pressurized fluid from the interior 30 to the surrounding environment to reduce the pressure differential or equalize the pressure within the interior 30 of the pressure chamber 26.

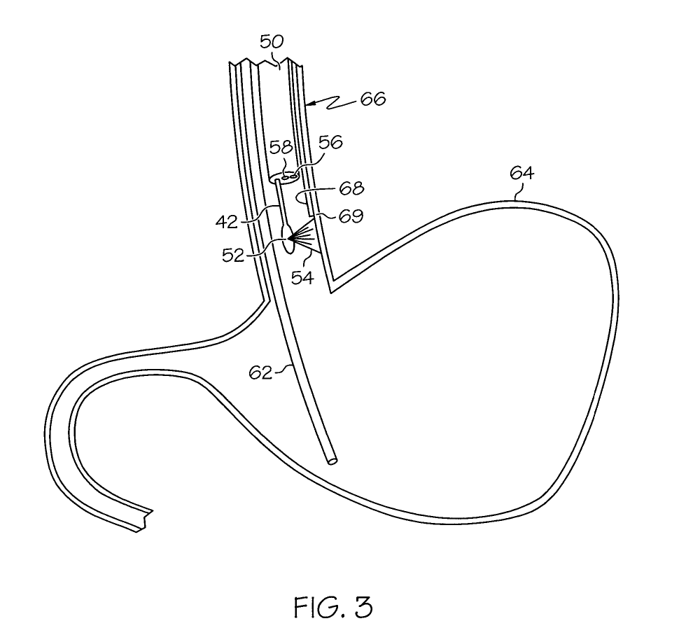

A valve 46, such as a solenoid valve, can control liquid flow from the pressure sensitive container 36. For instance, the valve 46 might comprise a solenoid valve operably connected to a foot control 48 such that a surgeon may conduct surgical procedures with hands-free control of liquid flow from the pressure sensitive container 36. As further illustrated in FIGS. 2 and 3, the device 10 might include an endoscope 50 that might house the tube 42. In certain embodiments, elements of the invention (e.g., the tube, etc.) May be endoscopically deployed through the lumens of conventional gastroscopes. Thus, existing endoscopes may be retrofitted to include the concepts of the present invention. As shown in FIG. 3, the tube 42 ends in a nozzle 52 adapted to disburse a high velocity liquid stream 54 against the interior surface of the esophagus 66. While the nozzle 52 is illustrated as disbursing a liquid stream in a single general direction, it is contemplated that the nozzle 52 may have a wide variety of disbursement patterns and might treat the entire interior peripheral surface of the esophagus simultaneously. Moreover, while a single nozzle 52 is shown, further embodiments may include a plurality of nozzles designed to disburse one or more liquid streams in one or more directions.

The endoscope 50, if provided, can also include an optical view port 56 and light source 58 adapted to permit viewing by the surgeon. A device might be provided to cooperate with the optical view port 56 to assist in observation by the surgeon. For example, the endoscope 50 may be provided with a monitor 60 or might include an optical viewing device adapted to provide visual output. Thus, the optical view port 56, if provided, allows the surgeon to view the treatment area by various output devices.

The device 10 can also include a tube 62 or other apparatus adapted to remove liquid from the stomach 64 or adjacent the treatment area. In one example, the tube 62 may be designed to remove liquid, gases and other material directly from an area of the stomach 64 as shown in FIG. 3. Although not shown, the device 10 might include an obstruction, such as an inflatable balloon to prevent entry of liquid into the stomach. In this example, the tube 62 might be designed to remove liquid and material in an area of the esophagus just above the obstruction.

In further embodiments, a high pressure pump or high pressure reservoir is provided in communication with a pressure chamber including a bag of saline solution in a chamber surrounded by pressurized gas. The pressure in the chamber may be adjusted to a selected pressure level, e.g., to several hundred pounds per square inch. The bag can be attached to a small diameter flexible tubing by a valve that regulates the flow of saline from the pressurized bag. The other end of the tubing is connected to a handle equipped with a trigger for remotely controlling the valve position (e.g., on/off). A flexible tubing of appropriate diameter can also extend from the handle several feet to a miniature nozzle head. The nozzle head can be perforated by strategically placed and angled jets of suitable size and configuration for safely and effectively ablating the esophageal mucosal layer 68 without significant injury to the submucosal layer 69. The jets, for example, can be deployed through a gastroscope into the esophageal lumen. The arrangement of the jets can also provide adequate coverage to enable sufficient mucosal layer resection without excessive splashing that could cause debris to obstruct the optics of the gastroscope.

The esophageal water-jet ablation exploits the difference in tissue structure between the esophageal mucosa 68 and the tough, collagen rich submucosal layers 69 to provide self limiting, selective ablation of the region affected by dysplastic and metaplastic columnar epithelium. The systems herein may also be used in combination with existing gastroscopes to allow easy practice of the invention in existing surgical environments. Moreover, the self limiting ablation procedure herein is limited without the application of high levels of surgical skill. Potentially, the procedures and apparatus herein can completely ablate mucosal linings during gastrointestinal surgical procedures while requiring only moderate surgical skills, without some of the complications typically associated with conventional surgical techniques such as stricture, perforation, or incomplete ablation.

An exemplary method of removing at least portions of a esophageal mucosal layer without significant injury to the submucosal layer will now be described. First, a nozzle 52 is positioned within the esophagus 66 adjacent the treatment area. Next, liquid is disbursed from the nozzle as a stream of liquid 54. The stream of liquid 54 is adapted to remove the esophageal mucosal layer 68 without significant injury to the submucosal layer 69 of the esophagus 66. Apparatus described herein may be useful to successfully treat Barrett's Esophagus as the esophageal mucosal layer 68 may be selectively removed without significant injury to the submucosal layer 69 of the esophagus 66. While apparatus described herein are disclosed as useful to remove the mucosal layer of the esophagus, it is contemplated that the apparatus herein may be employed with other gastrointestinal surgical procedures to ablate mucosal linings.

From the above description of the invention, those skilled in the art will perceive improvements, changes and modifications. Such improvements, changes and modifications within the skill of the art are intended to be covered by the appended claims.

What is claimed:

1. A method of treating an area of tissue of a patient with the area of tissue having a first tissue layer and a second tissue layer, comprising the steps of:
providing a liquid distribution source including a quantity of liquid;
providing a nozzle in liquid communication with the liquid distribution source;
positioning the nozzle with respect to the area of tissue; and
disbursing the quantity of liquid through the nozzle with a velocity sufficient to substantially remove the first tissue layer without significant injury to the second tissue layer.

2. The method of claim 1 wherein the area of tissue comprises an area of the esophagus.

3. The method of claim 2, wherein the first tissue layer comprises the mucosa layer of the esophagus and the second tissue layer comprises the submucosal layer of the esophagus.

4. The method of claim 1, wherein the method comprises treating intestinal columnar epithelium.

5. The method of claim 1, wherein the quantity of liquid comprises water.

6. The method of claim 1, wherein the quantity of liquid comprises a saline solution.

7. The method of claim 1, further comprising the steps of providing an endoscope with the nozzle and inserting an end of the endoscope with respect to the patient to position the nozzle with respect to the area of tissue.

8. The method of claim 7, wherein the endoscope comprises a gastroscope and the step of inserting comprises inserting an end of the endoscope through the mouth of the patient and into the esophagus of the patient.

9. A method of treating an esophagus of a patient comprising the steps of:
providing a liquid distribution source including a quantity of liquid;
providing a high-pressure source configured to apply pressure to the quantity of liquid;
providing a nozzle in liquid communication with the liquid distribution source;
positioning the nozzle with respect to the area of the esophagus; and
disbursing the quantity of liquid through the nozzle with a velocity sufficient to substantially remove a mucosa layer of the esophagus without significant injury to the submucosal layer of the esophagus.

10. A device for treating an area of tissue of a patient, comprising:
a liquid distribution source including a quantity of liquid;
a high-pressure source configured to apply pressure to the quantity of liquid; and
a nozzle in liquid communication with the quantity of liquid, wherein the device is configured to disburse the quantity of liquid through the nozzle with a velocity sufficient to substantially remove tissue from a patient.

11. A method using the device of claim 10 to treat an area of tissue of a patient with the area of tissue having a first tissue layer and a second tissue layer, the method comprising the steps of:
positioning the nozzle with respect to the area of tissue; and
disbursing the quantity of liquid through the nozzle with a velocity sufficient to substantially remove the first tissue layer without significant injury to the second tissue layer.

12. The method of claim 11, wherein the first tissue layer comprises the mucosa layer of an esophagus of the patient and the second tissue layer comprises the submucosal layer of the esophagus.

13. The device of claim 10, wherein the quantity of liquid comprises water.

14. The device of claim 10, wherein the quantity of liquid comprises a saline solution.

15. The device of claim 10, further comprising an endoscope including the nozzle.

16. The device of claim 15, wherein the endoscope comprises a gastroscope.

17. The device of claim 10, wherein the liquid distribution source comprises a pressure chamber including an interior provided with the quantity of liquid, wherein the high-pressure source is configured to pressurize the interior of the pressure chamber to apply pressure to the quantity of liquid.

18. The device fo claim 17, wherein the liquid distribution source comprises a pressure sensitive container located within the interior of the pressure chamber, wherein the quantity of liquid is contained within the pressure sensitive container.

19. The device of claim 10, wherein the liquid distribution source comprises a pressure sensitive container with the quantity of liquid contained within the pressure sensitive container, wherein the high-pressure source is configured to compress the pressure sensitive container to apply pressure to the quantity of liquid.

20. The device of claim 19, wherein the liquid distribution source comprises a pressure chamber including an interior area with the pressure sensitive container located within the interior area of the pressure chamber, wherein the high-pressure source is configured to pressurize the interior area of the pressure chamber to compress the pressure sensitive container.
